(19) United States
(12) Patent Application Publication
(10) Pub. No.: US 2025/0229135 A1
(43) Pub. Date: Jul. 17, 2025

(54) DEVICES, SYSTEMS, AND METHODS TO GENERATE EXERCISE RECOMMENDATIONS

(71) Applicant: iFIT Inc., Logan, UT (US)

(72) Inventor: Chase Brammer, Providence, UT (US)

(21) Appl. No.: 19/012,050

(22) Filed: Jan. 7, 2025

Related U.S. Application Data
(60) Provisional application No. 63/620,620, filed on Jan. 12, 2024.

(51) Int. Cl. A63B 24/00; G16H 10/60; G16H 20/30; G16H 80/00

(52) U.S. Cl. CPC A63B 24/0075; A63B 24/0062; G16H 10/60; G16H 20/30; G16H 80/00

(57) ABSTRACT

An exercise recommendation system may receive exercise information for a user. An exercise recommendation system may, based on the exercise information, identify a missed exercise activity. An exercise recommendation system may, based on the missed exercise activity and using an exercise chatbot, present the user with a query, the query including a request for additional information related to the missed exercise activity. An exercise recommendation system may receive a response to the query. An exercise recommendation system may apply a recommendation model to the response to generate an exercise recommendation.

### CROSS REFERENCE

This application claims the benefit and priority to U.S. Patent Application No. 63/620,620, filed Jan. 12, 2024, which is incorporated herein by reference in its entirety for all that it discloses.

### BACKGROUND

Health is a critical part of a person's well-being. People cultivate their health through health actions, including exercise, diet, lifestyle, and so forth. People may use exercise systems to facilitate their health journey. Conventional exercise systems may provide health recommendations to a person based on pre-determined responses to the person's requests. For example, a user that desires to lose weight and participate in a 5 kilometer race may receive a pre-determined diet and exercise system. But such pre-determined systems are inflexible. Indeed, such plans may be inapplicable to some user's lifestyle, attitude, motivational styles, and so forth.

### SUMMARY

In some aspects, the techniques described herein relate to a method for providing exercise recommendations. An exercise recommendation system receives exercise information for a user. Based on the exercise information, the exercise recommendation system identifies a missed exercise activity. Based on the missed exercise activity and using an exercise chatbot, the exercise recommendation system presents the user with a query. The query includes a request for additional information related to the missed exercise activity. The exercise recommendation system receives a response to the query. The exercise recommendation system applies a recommendation model to the response to generate an exercise recommendation.

In some aspects, the techniques described herein relate to a method for exercise recommendations. An exercise recommendation system receives exercise information for a user. Based on the exercise information, the exercise recommendation system provides the user with a query. The query requests health information from the user. The exercise recommendation system applies a health habit model to the health information. The health habit model identifies a health habit for the user. The exercise recommendation system generates, using the health habit model, a recommendation to improve the health habit. The exercise recommendation system presents presenting the recommendation to the user.

This summary is provided to introduce a selection of concepts that are further described below in the detailed description. This summary is not intended to identify key or essential features of the claimed subject matter, nor is it intended to be used as an aid in limiting the scope of the claimed subject matter. Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a representation of an exercise recommendation system, according to at least one embodiment of the present disclosure.

FIG. 2 is a representation of an exercise recommendation system, according to at least one embodiment of the present disclosure.

FIG. 3 is a representation of a string diagram of an exercise recommendation system, according to at least one embodiment of the present disclosure.

FIG. 4 is a representation of a string diagram of an exercise recommendation system, according to at least one embodiment of the present disclosure.

FIG. 5 is a flowchart of a method for providing exercise recommendations, according to at least one embodiment of the present disclosure.

FIG. 6 is a flowchart of a method for providing exercise recommendations, according to at least one embodiment of the present disclosure.

FIG. 7 is a schematic representation of a computing system, according to at least one embodiment of the present disclosure.

### DETAILED DESCRIPTION

This disclosure generally relates to generating and providing exercise and health recommendations for a user. An exercise recommendation system may receive exercise information for a user. The exercise recommendation system may identify various exercise activity trends and/or habit information from the exercise information. For example, the exercise recommendation system may identify a missed workout. An exercise chatbot may interact with the user to collect additional information regarding the missed workout. For example, the exercise chatbot may prepare or generate a query and provide the query to the user. The user may provide a response including additional information related to the missed workout. In some embodiments, the exercise chatbot engages with the user, such as by providing one or more follow-up queries. A recommendation model may analyze the additional information and prepare one or more recommendations for the user. The recommendations may be directed to facilitating the user not missing future workouts. In this manner, the exercise recommendation system may provide improved recommendations tailored to the user. This may help to improve the user's consistency in performing exercise activities.

In some embodiments, the exercise recommendation system may analyze the health habits of the user. For example, the exercise recommendation system may receive and analyze historical health information for the user. A health habit model may analyze the historical health information for the user. The health habit model may prepare one or more recommendations to improve the user's health habits. For example, the health habit model may prepare a recommendation to instruct the user on actions that he or she may perform to improve health habits. The exercise recommendation system may provide the user with the recommendation. In some embodiments, the exercise recommendation system may follow up on the user's exercise information to determine whether the user has adopted the recommendation and generated the desired habit. In this manner, the exercise recommendation system may continue to monitor the user's exercise information and provide recommendations to improve the user's health habits.

As illustrated by the foregoing discussion, the present disclosure utilizes a variety of terms to describe features and advantages of the exercise recommendation system. Additional detail is now provided regarding the meaning of such terms. For example, as used herein, the term “exercise information” (e.g., health information) refers to information related to health and/or exercise. In particular, the term exercise information may include information related to one or more exercise activities (e.g., workouts). For example, exercise information may include information related to the performance of the exercise activity, such as exercise activity type, exercise activity day (e.g., date, day of the week), exercise activity time of day, exercise device used, exercise device operating parameters (e.g., resistance, speed, incline level, weight setting), location of exercise device, exercise activity duration, training plan information, any other information related to the performance of the exercise activity, and combinations thereof. In some embodiments, exercise information includes user exercise information. For example, the exercise information may include heartrate information, electrocardiogram (EKG) information, blood sugar information, blood oxygen information, any other user exercise information, and combinations thereof. In some embodiments, exercise information includes user lifestyle or habit information. For example, user lifestyle or habit information may include sleep information (e.g., duration, time, quality), diet and nutrition information (e.g., food eaten, supplements taken, time of meals), work details, any other user lifestyle or habit information, goal information, and combinations thereof. In some embodiments, exercise information includes qualitative user exercise information. For example, qualitative user exercise information may include stress levels, pain levels, fatigue levels, attitude levels, motivation levels, any other qualitative user exercise information, and combinations thereof.

As used herein, a “foundation model” refers to an artificial intelligence (AI) or machine learning (ML) model that is trained to generate an output in response to an input based on a large dataset. The present disclosure may interchangeably refer to foundation models as AI models or ML models. A foundation model may be formed using a neural network having a significant number of parameters (e.g., billions of parameters). The foundation model may utilize the parameters to perform a task or otherwise generate an output based on an input. In one or more embodiments described herein, a foundation model is trained to generate a response to a query. In some implementations, a foundation model refers to a large language model (LLM). The foundation model be trained in any manner. For example, the foundation model may be trained on pattern recognition and text prediction. For example, the foundation model may be trained to predict the next word of a particular sentence or phrase. In one or more implementations described herein, the foundation model refers specifically to an LLM, though other types of foundation models may be used in generating responses to input queries.

As used herein, a chatbot may be a foundation model or an ML model that is trained in natural language algorithms to provide queries to a user and receive responses to the queries. The chatbot may be trained in natural language algorithms that may simulate a human interaction. For example, the chatbot may be trained to generate and present a query using syntax, verbs, nouns, and other grammatical elements to provide information and request information as a human being would. The chatbot may be trained to collect information based on an input dataset, such as the exercise information discussed herein. In some embodiments, the chatbot analyzes the input dataset, identify initial patterns in the input dataset, and request additional information to complete the pattern analysis. In some embodiments, the chatbot receives the pattern analysis from a different ML model, such as an exercise analysis model, health habit model, or other ML model. The chatbot may be interactive. For example, the chatbot may be trained to analyze the received response and generate additional content to provide the user. Such content may include recommendations, additional queries, motivational information, any other content, and combinations thereof.

As used herein, a recommendation model may refer to a foundation model that is trained to generate health or exercise recommendations based on an input dataset. The input dataset may include exercise information and/or historical exercise information. Historical exercise information may include any exercise information previously collected. In some embodiments, historical exercise information includes exercise information related to exercise activities prior to the most recent exercise activities. In some embodiments, historical exercise information includes daily exercise information for a period of time (e.g., one day, one week, one month, one year, multiple years). The recommendation model may be trained on a recommendation training dataset. The recommendation training dataset may include exercise information from people that exhibit positive exercise habits and/or have met previously set exercise goals or health habit goals. The recommendation model may receive the exercise information for a user and compare the user's exercise information, exercise goals, and health habit goals to the patterns identified by the recommendation model. The recommendation model may provide behavioral changes for the user to implement to meet his or her goals.

As used herein, an exercise recommendation may be used to refer to any recommendation to improve a user's health, including a recommendation to improve health habits and/or exercise consistency. For example, the exercise recommendation may include a change in behavior that may be associated with a user's health habits. In some examples, the exercise recommendation may include a change in environment. In some examples, the exercise recommendation may include any change in time of day for exercise, a change in exercise activity duration, a change in exercise activity intensity, a change in trainer, a change in exercise activity location, any other change in behavior or environment, and combinations thereof.

In some embodiments, the exercise recommendation is informational. For example, the exercise recommendation may include environmental information, habit stacking, a reward cycle, educational material, any other information, and combinations thereof. The environmental information may include any type of environmental information, such as locations for exercise, exercise apparel, people to exercise with, music type, music playlists, trainer ID, any other environmental information, and combinations thereof. In some examples, the educational material may include process-oriented education (e.g., changes in a series of activities leading to and/or during an exercise activity). In some examples, the educational material may include consistency education.

As used herein, the term “health habits” may refer to actions that are consistently performed that result in outcomes associated with positive health. Health habits may refer to any type of behavior, such as sleep-related actions, exercise activity-related actions, food and meal-related actions, any other health actions, and combinations thereof. In some embodiments, health habits are different for different people. In some embodiments, the recommendation model identifies commonalities in the exercise information and health habits, and provide recommendations to change the health habits. A health habit model may be an ML model that may be trained on trainer health habits of trainers.

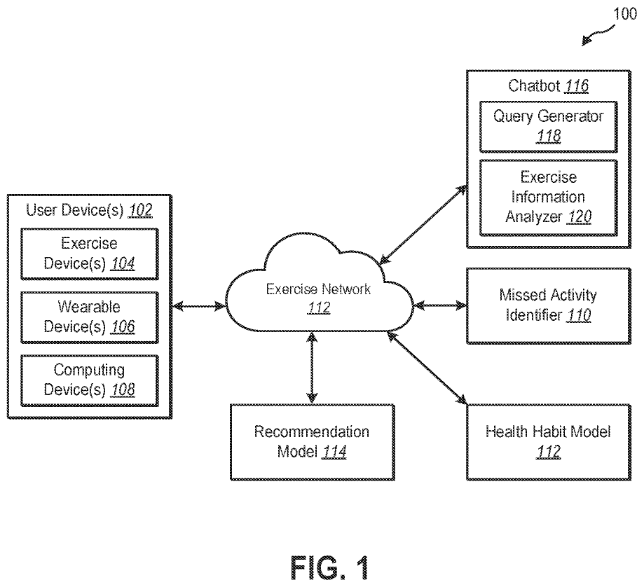

FIG. 1 is a representation of an exercise recommendation system 100, according to at least one embodiment of the present disclosure. The exercise recommendation system 100 may generate and prove exercise and health recommendations to the user based on exercise information collected by and from the user. The exercise recommendation system 100 may collect exercise and health information from the user using one or more user devices 102. The user devices 102 may include any type of user device. For example, the user devices 102 may include exercise devices 104, such as treadmills, elliptical machines, stationary bicycles, rowers, cable exercise devices, weight devices, any other exercise device 104, and combinations thereof. The user devices 102 may include one or more wearable devices 106. The wearable devices 106 may be any type of wearable device, such as a smartwatch, a smart ring, a sleep monitor, a heartrate monitor, any other type of wearable device 106, and combinations thereof. The user devices 102 may include a computing device 108, such as a mobile device, a smartphone, a tablet, a laptop computer, a desktop computer, a server computer, any other computing device 108, and combinations thereof. In some embodiments, the user devices 102 include any other type of device, such as medical devices, GPS trackers, pedometers, any other user devices 102, and combinations thereof.

The exercise recommendation system 100 may provide the exercise information with one or more ML models, such as a missed activity identifier 110, a health habit model 111, a recommendation model 114, and an exercise chatbot 116. The ML models may analyze the exercise information to provide recommendations to the user. For example, the missed activity identifier 110 may analyze the exercise information and identify a missed exercise activity. To identify the missed activity, the missed activity identifier 110 may identify patterns of exercise activities and determine that an exercise activity was not performed based on the identified pattern and the exercise information. In some examples, the health habit model 111 may analyze the exercise information to identify health habits for the user. The health habit model 111 may compare the user's health habits to health habits identified by the health habit model 111. The exercise recommendation system 100 may apply the recommendation model 114 to generate exercise recommendations. For example, the recommendation model 114 may receive the exercise information, the missed activity, and the health habit model 111 and prepare or generate one or more recommendations for the user to implement or change a behavior to improve the user's health and/or exercise consistency. This may help the user to make and attain his or her health and exercise goals.

The user devices 102 may be in communication with the exercise chatbot 116, the missed activity identifier 110, the health habit model 111, and the recommendation model 114 over an exercise network 112. The exercise network 112 may be any type of network. For example, the exercise network 112 may be a local area network (LAN), a wide area network (WAN), a Wi-Fi network, a cellular network, any other network, and combinations thereof.

The exercise chatbot 116 may interact with the user. For example, the exercise chatbot 116 may include a query generator 118 that generates and provides queries to the user to request additional information from the user. In some embodiments, the exercise chatbot 116 includes an exercise information analyzer 120 that may analyze the exercise information and/or the additional information from the user.

The query generator 118 may generate queries to provide to the user. The query generator 118 may generate the queries based on any information. For example, the query generator 118 may generate the queries based on the exercise information. In some examples, the query generator 118 may generate the queries based on missed activity information received from the missed activity identifier 110. In some examples, the query generator 118 may generate the queries based on health habit information received from the health habit model 111. In some examples, the query generator 118 may generate the queries based on recommendation information from the recommendation model 114. In some examples, the query generator 118 may generate the queries based on pre-determined lines of inquiry. The pre-determined lines of inquiry may include goals, attitudes, interests, access to facilities, access or ownership of equipment, time availability, past successes, any other pre-determined line of inquiry, and combinations thereof.

The user may interact with the exercise chatbot 116 on one or more of the user devices 102. For example, the exercise chatbot 116 may present the query to the user through one of the user devices 102 (e.g., through text, images, and/or videos a display, through sound from a speaker). The exercise chatbot 116 may provide a response to the query through the user device 102. The user may provide the response to the exercise chatbot 116 in any manner. For example, the user may provide the response to the through text input, image input (e.g., pictures, illustrations), video input, sound input (e.g., voice commands, text-to-speech), any other input mechanism, and combinations thereof. In some examples, the user may provide the response by uploading or linking a dataset. For example, the user may include health information in a datafile and upload the datafile to the exercise chatbot 116. In some examples, the user may have recorded health information using a third-party application, and the user may provide a link, download, or other mechanism to access the recorded data from the third-party application.

In some embodiments, the user provides exercise information to the exercise chatbot 116 that is the same as at least a portion of the exercise information collected by the user devices 102. This may allow the exercise chatbot 116 to validate the exercise information and/or ensure that the exercise information provided by the user devices 102 is accurate, complete, up-to-date, or representative of the user's actual exercise information.

In accordance with at least one embodiment of the present disclosure, the exercise chatbot 116 requests additional exercise information from the user. The additional exercise information include exercise information that was not included in the originally provided exercise information. For example, the additional exercise information may include exercise information that the user did not previously record on the user devices 102. In some examples, the additional exercise information may include qualitative exercise information that was previously uncaptured. In some examples, the additional exercise information may include sleep quality, a cause of the missed exercise activity, schedule information, exercise activity quality information, exercise activity difficulty information, exercise information intensity information, habit information, motivational information, any other exercise information, and combinations thereof.

In some embodiments, the exercise chatbot 116 requests the additional exercise information in the query. For example, the query generator 118 may generate the query and request the additional exercise information. The exercise chatbot 116 may provide the query to the user. The user may prepare and submit a response to the exercise chatbot 116 with the additional exercise information.

In some embodiments, the exercise chatbot 116 provides the additional information to any other part of the exercise recommendation system 100. For example, the exercise chatbot 116 may provide the additional exercise information to the missed activity identifier 110, the health habit model 111, the recommendation model 114, any other portion of the exercise recommendation system 100, and combinations thereof. This may allow the exercise recommendation system 100 to identify additional patterns or associations between the originally provided exercise information, the additional exercise information, the training plan information, any other information, and combinations thereof.

The query generator 118 may generate the query for the additional information based on any parameter. For example, the query generator 118 may generate the query based on a set of pre-questions. In some examples, the query generator 118 may generate the query based on the originally provided exercise information. In some examples, the query generator 118 may generate the query based on an analysis from the missed activity identifier 110, the health habit model 111, the recommendation model 114, any other portion of the exercise recommendation system 100, and combinations thereof.

In some embodiments, the exercise chatbot 116 enters into a conversation with the user. For example, the exercise chatbot 116 may receive the response including the additional exercise information from the user. The exercise information analyzer 120 may analyze the received additional exercise information. Based on the analysis of the additional exercise information, the query generator 118 may generate a follow-up query for the user asking for further exercise information. The follow-up query may be related to the received additional exercise information provided by the user as the response to the first query.

The exercise chatbot 116 may send the follow-up query to the user, and the user may provide follow-up exercise information based on the follow-up query. In this manner, the user may enter into a chat or a conversation with the user. The exercise chatbot 116 may provide any number of follow-up queries to the user, and the user may provide any number of responses. In this manner, the exercise chatbot 116 may receive more exercise information with which the exercise recommendation system 100 may prepare exercise recommendations.

In some embodiments, the follow-up query includes a response to a question from the user. For example, in the response to one or more of the queries, the user may provide a question to the exercise chatbot 116. The question may be any type of question, such as a request for additional information, a request for clarification, a health question related to health information, an exercise information question related to a particular piece of exercise information or set of exercise information, an exercise activity question related to a particular exercise activity, an exercise device question related to a particular exercise device, any other request for information, and combinations thereof.

In some embodiments, the follow-up query is processed or generated using natural-language processing. For example, the query generator 118 may include an ML model trained to generate natural language responses. This may help to improve the responsiveness of the exercise chatbot 116 and/or improve the engagement of the user with the exercise chatbot 116.

In some embodiments, the query generator 118 generates the follow-up query based on information received from the missed activity identifier 110, the health habit model 111, and/or the recommendation model 114. For example, the user may provide additional exercise information based on a query from the exercise chatbot 116. The missed activity identifier 110 may identify one or more potential explanations for a missed exercise activity, and the missed activity identifier 110 may have the query generator 118 generate a follow-up query to request additional from the user related to the potential explanation for the missed exercise activity. The exercise chatbot 116 may provide the follow-up query to the user, and the user may provide the additional exercise information related to the follow-up query. This may allow the missed activity identifier 110 to enhance its analysis of the exercise information to identify the potential explanation for the missed activity. In some examples, the recommendation model 114 may compare the missed exercise activity with a previous missed exercise activity to identify common behaviors in the missed exercise activity and the previous missed exercise activity.

In some examples, the query generator 118 may generate the follow-up query based on the health analysis by the health habit model 111. For example, the health habit model 111 may identify a potential health habit from the exercise information and/or the health information provided by the user. The health habit model 111 may request additional information, and the query generator 118 may generate the follow-up query based on the requested additional information. Exercise chatbot 116 may provide the follow-up query to the user based on the request for additional information. The user may provide the additional exercise information based on the follow-up query. This may allow the health habit model 111 to enhance its analysis of the exercise information to identify one or more health habits of the user.

In some examples, the query generator 118 may generate the follow-up query based on a recommendation analysis by the recommendation model 114. For example, the recommendation model 114 may begin preparing an exercise recommendation. The recommendation model 114 may request additional information about the user from the exercise chatbot 116. The query generator 118 may generate the follow-up query based on the requested additional information. The user may provide the additional exercise information based on the follow-up query. This may allow the recommendation model 114 to enhance its recommendation based on the additional exercise information received by the user.

In accordance with at least one embodiment of the present disclosure, the exercise chatbot 116 presents the query to the user without any user input. For example, the exercise chatbot 116 may proactively send the user the query without receiving a user question or request for information. In some examples, the exercise chatbot 116 may proactively send the user the query based on one or more of the analyses of the missed activity identifier 110, the health habit model 111, the recommendation model 114, and combinations thereof. In some examples, the exercise chatbot 116 may proactively present the user with the query at a pre-scheduled time. The pre-scheduled time may be based on any time. For example, the pre-scheduled time may include a time of day, a day of the week, a day of the month, any other pre-scheduled time, and combinations thereof. In some embodiments, the pre-scheduled time is based on a trigger event. For example, the pre-scheduled time may be based on after completing an exercise activity, in a period after a missed exercise activity, after identifying a health habit, after identifying an area for improvement of a health habit, any other trigger event, and combinations thereof.

As a specific, non-limiting example, the missed activity identifier 110 may identify, using the exercise information, that the user has missed an activity. The missed activity identifier 110 may provide the exercise information analyzer 120 and/or the query generator 118 the missed activity. In some examples, the missed activity identifier 110 may provide the recommendation model 114 with the missed exercise activity and the recommendation model 114 may prepare a recommendation for the user. Upon receipt of the missed activity and/or the recommendation based on the missed activity, the exercise chatbot 116 may generate a query. The query may include any information discussed herein, including a request for additional exercise information, motivational information, some or all of the recommendation by the recommendation model 114, a request for additional information by the missed activity identifier 110 and/or the recommendation model 114, any other information, and combinations thereof. As discussed herein, the exercise chatbot 116 may provide the query to the user and receive a response from the user. In some examples, the exercise chatbot 116 may enter into a conversation with the user based on the missed activity. This may help the user to identify the missed activity and implement changes to his or her lifestyle to prevent the missing another activity.

As a specific, non-limiting example, the health habit model 111 may receive exercise activity information (e.g., health habit information). The health habit model 111 may identify one or more existing health habits or opportunities for making and/or improving of a health habit. The health habit model 111 may provide the health habit and/or the opportunities to the exercise chatbot 116. In some examples, the health habit model 111 may provide the health habit and/or the opportunities to the recommendation model 114, and the recommendation model 114 may provide a recommendation to the exercise chatbot 116. The exercise chatbot 116 may generate and/or present a query to the user, the query including the health habit and/or the recommendation. As discussed herein, the user may prepare a response. In some examples, the exercise chatbot 116 may enter into a conversation with the user based on the health habit and/or opportunities. This may help the user to identify health habits and/or make or improve new health habits.

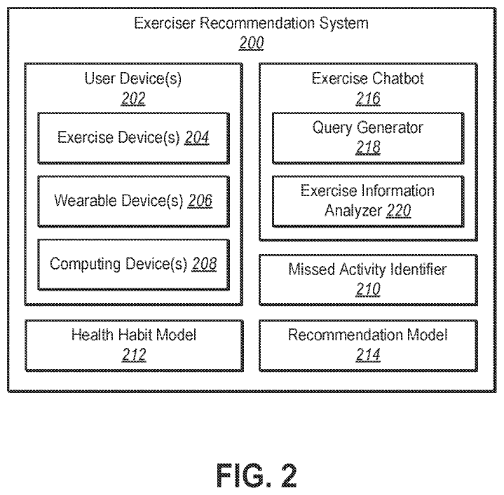

FIG. 2 is a representation of an exercise recommendation system 200, according to at least one embodiment of the present disclosure. Each of the components of the exercise recommendation system 200 can include software, hardware, or both. For example, the components can include one or more instructions stored on a computer-readable storage medium and executable by processors of one or more computing devices, such as a client device or server device. When executed by the one or more processors, the computer-executable instructions of the exercise recommendation system 200 can cause the computing device(s) to perform the methods described herein. Alternatively, the components can include hardware, such as a special-purpose processing device to perform a certain function or group of functions. Alternatively, the components of the exercise recommendation system 200 can include a combination of computer-executable instructions and hardware.

Furthermore, the components of the exercise recommendation system 200 may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

The exercise recommendation system 200 may include one or more user devices 202. The user may interact with the user devices 202 to communicate with the exercise recommendation system 200. For example, the user may input exercise information into the user devices 202. In some examples, the user may use the user devices 202 to collect exercise information. The user devices 202 may include one or more sensors, and the sensors on the user devices 202 may collect exercise information about the user. As discussed herein, the user devices 202 may include any type of user device, including exercise devices 204, wearable devices 206, computing devices 208, any other user device 202, and combinations thereof.

The exercise recommendation system 200 may further include an exercise chatbot 216. The exercise chatbot 216 may communicate with the user regarding exercise information. For example, the exercise chatbot 216 may present the user with queries. The user may respond to the queries to the exercise chatbot 216. As discussed herein, the exercise chatbot 216 may provide multiple queries to the user and the user may respond to the multiple queries. In this manner, the exercise chatbot 216 may hold a conversation or other back-and-forth engagement with the user.

The exercise chatbot 216 may include a query generator 218. The query generator 218 may be trained to generate queries for the user. For example, the query generator 218 may be trained in natural-language processing to provide queries to the user in a manner that the user may easily understand and be comfortable engaging in. In some examples, the query generator 218 may be trained on exercise information, as discussed herein.

The exercise chatbot 216 may further include an exercise information analyzer 220. The exercise information analyzer 220 may analyze exercise information, including exercise information received by the user. The exercise information analyzer 220 may communicate with the query generator 218 to prepare the queries.

The exercise recommendation system 200 may further include a missed activity identifier 210. The missed activity identifier 210 may receive the exercise information from the user devices 202 and/or additional exercise information from the exercise chatbot 216. The missed activity identifier 210 may identify whether the user missed an activity. If the user misses an activity, the missed activity identifier 210 may communicate the missed activity to the exercise chatbot 216. The exercise chatbot 216 may generate and present a query to the user based on the missed activity.

The exercise recommendation system 200 may include a health habit model 212. The health habit model 212 may receive the exercise information from the user devices 202 and/or additional exercise information from the exercise chatbot 216. The health habit model 212 may analyze the exercise information and identify health habits and/or opportunities for improved or new health habits. The health habit model 212 may provide the health habits and/or the opportunities to the exercise chatbot 216. As discussed herein, the exercise chatbot 216 may generate and present a query to the user based on the health habits and/or opportunities.

The exercise recommendation system 200 may further include a recommendation model 214. The exercise recommendation system 200 may apply the recommendation model 214 to the exercise information. For example, the recommendation model 214 may receive the exercise information from the user devices 202 and/or additional exercise information from the exercise chatbot 216. In some embodiments, the recommendation model 214 receives exercise analyses from the missed activity identifier 210 and/or the health habit model 212. The recommendation model 214 may be trained to generate an exercise recommendation for the user. The recommendation model 214 may provide the exercise recommendation to the user devices 202. In some examples, the recommendation model 214 may provide the recommendation to the exercise chatbot 216 and the exercise chatbot 216 may provide the recommendation to the user as a query and/or as part of a conversation with the user.

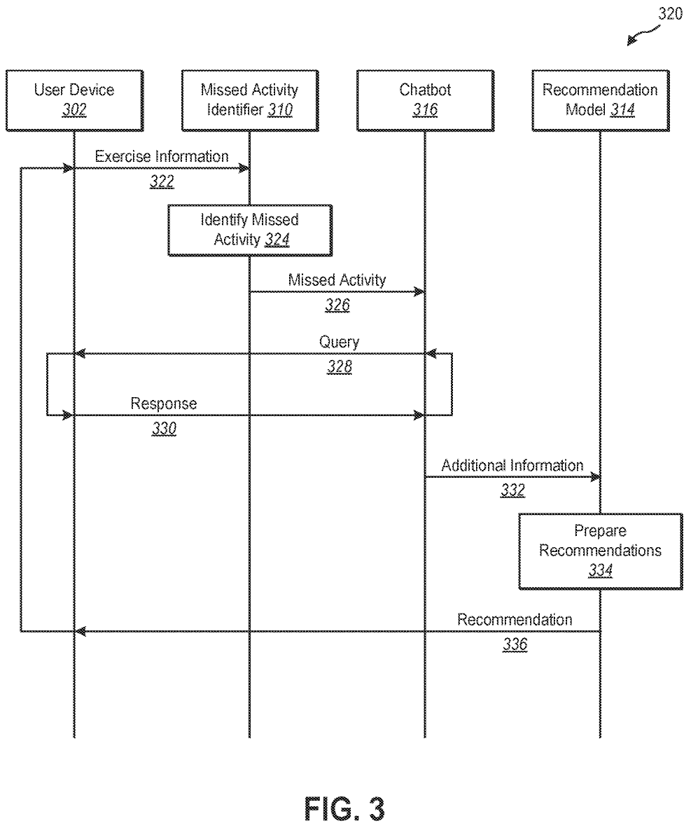

FIG. 3 is a representation of a string diagram 320 of an exercise recommendation system, according to at least one embodiment of the present disclosure. A user may provide exercise information 322 to an exercise recommendation system from a user device 302. A missed activity identifier 310 may receive the exercise information 322. The missed activity identifier 310 may identify a missed activity 326 in the exercise information 322. For example, the missed activity identifier 310 may include an ML, model that may be trained to identify 324 whether the user has missed an activity.

An exercise chatbot 316 may receive the exercise information 322 and the missed activity 326. The exercise chatbot 316 may analyze the missed activity 326 and the exercise information 322 and prepare a query 328 for the user. The exercise chatbot 316 may generate the query 328 in any manner. For example, the exercise chatbot 316 may generate the query 328 requesting additional information based on the missed activity 326 and/or the exercise information 322. The exercise chatbot 316 may provide the query 328 to the user at the user device 302. The user may prepare a response 330 including additional exercise information. For example, the response 330 may include the user's response to any questions or requests in the query 328.

The user device 302 may provide the response 330 to the exercise chatbot 316. As discussed herein, the exercise chatbot 316 may receive the response 330 and prepare a follow-up query 328. The exercise chatbot 316 may provide the follow-up query 328 to the user at the user device 302, and the user may provide a follow-up response 330 or an additional response 330 to the exercise chatbot 316. This loop may be repeated any number of times until the exercise chatbot 316 does not have any additional queries 328 to provide the user. While embodiments of the present disclosure have discussed the exercise chatbot 316 as providing the follow-up query 328 in response to the response 330 by the user, it should be understood that the exercise chatbot 316 may provide more queries 328 than the user provides responses 330. For example, the exercise chatbot 316 may provide the follow-up query 328 without a response 330 from the user. In some examples, the user may provide more responses 330 than the user receives queries 328.

In some embodiments, the user provides a response 330 to the exercise chatbot 316 without receiving a query 328. For example, the user provide the exercise chatbot 316 with a question or a request for a recommendation and/or additional information. The user may provide the question or request as a response 330 to a previous query 328. For example, the exercise chatbot 316 may include a window having one or more text entry boxes. The window may include a history of the conversation with the user. The user may provide the question or request in the text entry box. The exercise chatbot 316 may receive the question or request as a response 330. The exercise chatbot 316 may then initiate a conversation with the user based on the user input.

The exercise chatbot 316 may provide the additional information 332 received by the user device 302 to a recommendation model 314. The exercise recommendation system may apply the recommendation model 314 to the exercise information 322. The recommendation model 314 may further receive the additional exercise information 332 from the user device 302. The additional exercise information 332 may include the additional exercise information provided in the responses 330. The exercise chatbot 316 provides the additional information 332 to the recommendation model 314 at any point in time. For example, the exercise chatbot 316 may provide the additional exercise information 332 to the recommendation model 314 as soon as it receives a response 330 from the user device 302. In some examples, the exercise chatbot 316 may combine one or more responses 330 and provide them to the additional exercise information 332 to the recommendation model 314 as a group or a bundle. This may help to reduce bandwidth of the exercise recommendation system.

As discussed herein, the exercise recommendation system may apply the recommendation model 314 to the exercise information 322. The recommendation model 314 may prepare 334 a recommendation 336 for the user. The recommendation 336 may be based on the exercise information 322 and/or the additional exercise information 332. As discussed herein, the recommendation 336 may include a change in one or more behaviors that may help the user to not miss a future exercise activity, or to be more reliable or regular in attending future exercise activities. The recommendation model 314 may send the recommendation 336 to the user device 302.

In accordance with at least one embodiment of the present disclosure, after the user device 302 receives the recommendation 336, the user device 302 transmits exercise information 322 to the missed activity identifier 310, the exercise chatbot 316, and the recommendation model 314, and the techniques described herein are repeated. For example, the recommendation 336 may include a recommendation to change a lifestyle action, such as an exercise time. The exercise information 322 may include information about the exercise time of the user. The missed activity identifier 310 may analyze the exercise information 322 to determine whether the user missed an exercise activity based on the new exercise time. The exercise chatbot 316 may provide the user with information regarding the new exercise time and the recommendation 336. In some embodiments, the missed activity identifier 310 identifies a success, or identifies when the user successfully develops a habit or exhibits an improved pattern of behavior based on the recommendation 336.

In some examples, the exercise information 322 may indicate that the recommendation 336 was not effective at encouraging the user to not miss exercise activities. For example, the missed activity identifier 310 may analyze the exercise information 322 after the user receives the recommendation 336. The missed activity identifier 310 may identify that the user missed another activity. In some embodiments, the missed activity identifier 310, the exercise chatbot 316, and/or the recommendation model 314 identifies improvement or backsliding based on the exercise information 322 and/or additional exercise information 332 provided based on a query 328 to the user. For example, the exercise recommendation system may apply the missed activity identifier 310 and/or the recommendation model 314 to the exercise information and/or the additional exercise information 332. The recommendation model 314 may analyze the exercise information 322 and/or the additional exercise information 332 and prepare another recommendation 336 to facilitate the user not missing an additional exercise activity, or for the user to be more consistent in exercising.

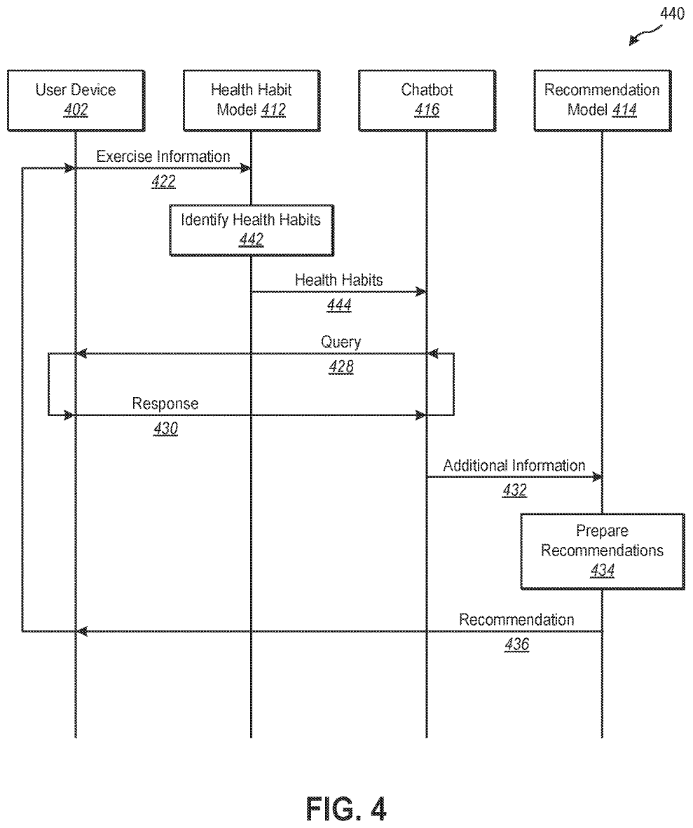

FIG. 4 is a representation of a string diagram 440 of an exercise recommendation system, according to at least one embodiment of the present disclosure. A user may provide exercise information 422 to an exercise recommendation system from a user device 402. A health habit model 412 may receive the exercise information 422. The health habit model 412 may identify 442 health habits 444 in the exercise information 422. For example, the health habit model 412 may include an ML model that may be trained to identify 442 patterns of behavior correlated with health outcomes.

The health habit model 412 may provide health habits 444 to an exercise chatbot 416. The exercise chatbot 416 may further receive the exercise information 422 from the user device 402. Based on the health habits 444 and/or the exercise information 422, the exercise chatbot 416 may generate and present a query 428 to the user at the user device 402. The user may receive the query 428 and send a response 430 including additional exercise information 432 to the exercise chatbot 416. The exercise chatbot 416 may provide the additional exercise information 432 to a recommendation model 414.

The recommendation model 414 may receive the additional exercise information 432. In some embodiments, the recommendation model 414 receives the exercise information 422 from the user device 402. The recommendation model 414 may prepare 434 an exercise recommendation 436 for the user. The recommendation model 414 may send the exercise recommendation 436 to the user device 402. As discussed herein, the exercise recommendation 436 may include any recommendation. For example, the exercise recommendation 436 may include a recommendation for meal planning. This may help the user to prepare meals ahead of time and encourage health diet habits.

In accordance with at least one embodiment of the present disclosure, after the user receives the exercise recommendation 436, the health habit model 412 receives exercise information 422. The health habit model 412 may identify whether the exercise recommendation 436 has been implemented. The techniques described herein may be applied to the exercise information 422 after receiving and/or implementing the exercise recommendation 436. This may facilitate the health habit model 412, the exercise chatbot 416, and/or the recommendation model 414 understanding whether the user implemented the exercise recommendation 436. The exercise chatbot 416 may engage with the user regarding whether the user implemented the exercise recommendation 436. For example, the exercise chatbot 416 may provide encouragement and/or follow-up questions regarding the implementation of the exercise recommendation 436. In some examples, the recommendation model 414 may prepare 434 additional or adjusted recommendations to further improve the user's health habits.

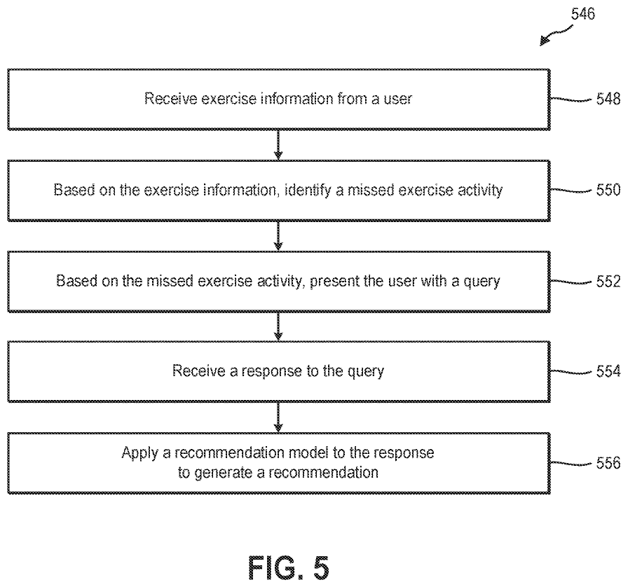

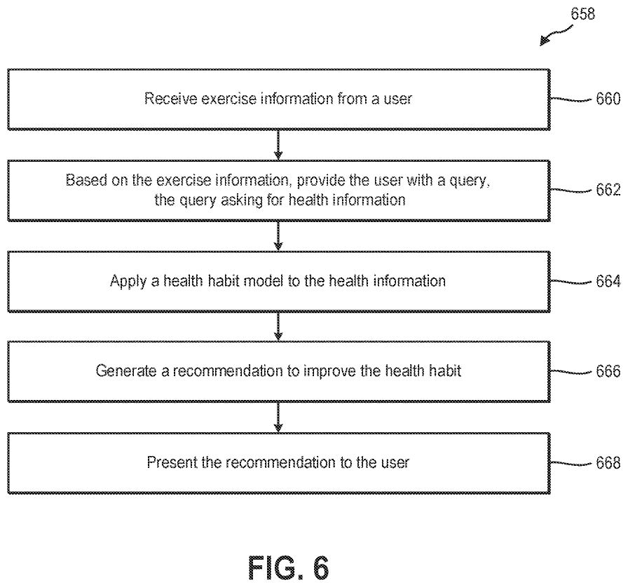

FIGS. 5 and 6, the corresponding text, and the examples provide a number of different methods, systems, devices, and computer-readable media of the exercise recommendation system 100. In addition to the foregoing, one or more embodiments can also be described in terms of flowcharts comprising acts for accomplishing a particular result, as shown in FIGS. 5 and 6. FIGS. 5 and 6 may be performed with more or fewer acts. Further, the acts may be performed in differing orders. Additionally, the acts described herein may be repeated or performed in parallel with one another or parallel with different instances of the same or similar acts.

As mentioned, FIG. 5 illustrates a flowchart of a series of acts or a method 546 for providing exercise recommendations, according to at least one embodiment of the present disclosure. While FIG. 5 illustrates acts according to one embodiment, alternative embodiments may omit, add to, reorder, and/or modify any of the acts shown in FIG. 5. The acts of FIG. 5 can be performed as part of a method. Alternatively, a computer-readable medium can comprise instructions that, when executed by one or more processors, cause a computing device to perform the acts of FIG. 5. In some embodiments, a system can perform the acts of FIG. 5.

An exercise recommendation system may receive exercise information for a user at 548. Based on the exercise information the exercise recommendation system may identify a missed exercise activity at 550. Based on the missed exercise activity, and using an exercise chatbot, the exercise recommendation system may present the user with a query at 552. The query may ask for additional information related to the missed exercise activity. The exercise recommendation system may receive a response to the query from the user at 554. The exercise recommendation system may apply a recommendation model to the response and/or the exercise information to generate an exercise recommendation at 556.

As mentioned, FIG. 6 illustrates a flowchart of a series of acts or a method 658 for providing exercise recommendations, according to at least one embodiment of the present disclosure. While FIG. 6 illustrates acts according to one embodiment, alternative embodiments may omit, add to, reorder, and/or modify any of the acts shown in FIG. 6. The acts of FIG. 6 can be performed as part of a method. Alternatively, a computer-readable medium can comprise instructions that, when executed by one or more processors, cause a computing device to perform the acts of FIG. 6. In some embodiments, a system can perform the acts of FIG. 6.

An exercise recommendation system may receive exercise information for a user at 660. Based on the exercise information, the exercise recommendation system may provide the user with a query at 662. The query may ask for health information. The exercise recommendation system may receive health information from the user. The health information include additional exercise information for the user. The exercise recommendation system may apply a health habit model to the health information at 664. The heath habit model may identify a health habit for the user. The exercise recommendation system may generate, using the health habit model and/or a recommendation model, a recommendation for the user at 666. The recommendation may be to improve the health habit of the user. The exercise recommendation system may then present the recommendation to the user at 668.

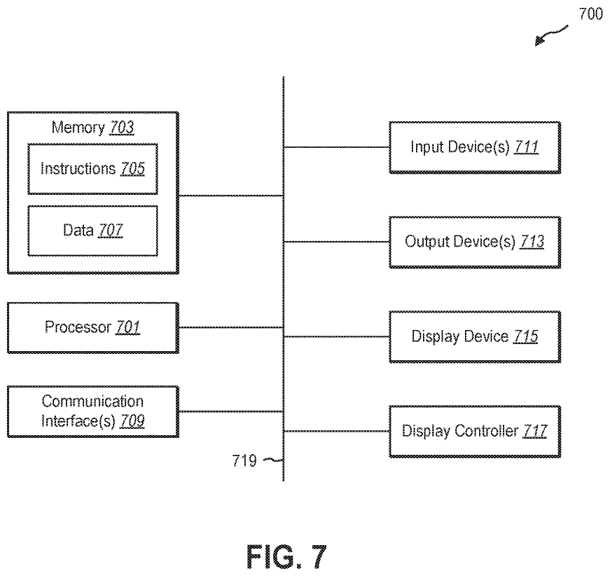

FIG. 7 illustrates certain components that may be included within a computer system 700. One or more computer systems 700 may be used to implement the various devices, components, and systems described herein.

The computer system 700 includes a processor 701. The processor 701 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 701 may be referred to as a central processing unit (CPU). Although just a single processor 701 is shown in the computer system 700 of FIG. 7, in an alternative configuration, a combination of processors (e.g., an ARM and DSP) could be used.

The computer system 700 also includes memory 703 in electronic communication with the processor 701. The memory 703 may be any electronic component capable of storing electronic information. For example, the memory 703 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 705 and data 707 may be stored in the memory 703. The instructions 705 may be executable by the processor 701 to implement some or all of the functionality disclosed herein. Executing the instructions 705 may involve the use of the data 707 that is stored in the memory 703. Any of the various examples of modules and components described herein may be implemented, partially or wholly, as instructions 705 stored in memory 703 and executed by the processor 701. Any of the various examples of data described herein may be among the data 707 that is stored in memory 703 and used during execution of the instructions 705 by the processor 701.

A computer system 700 may also include one or more communication interfaces 709 for communicating with other electronic devices. The communication interface(s) 709 may be based on wired communication technology, wireless communication technology, or both. Some examples of communication interfaces 709 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 700 may also include one or more input devices 711 and one or more output devices 713. Some examples of input devices 711 include a keyboard, mouse, microphone, remote control device, button, joystick, trackball, touchpad, and lightpen. Some examples of output devices 713 include a speaker and a printer. One specific type of output device that is typically included in a computer system 700 is a display device 715. Display devices 715 used with embodiments disclosed herein may utilize any suitable image projection technology, such as liquid crystal display (LCD), light-emitting diode (LED), gas plasma, electroluminescence, or the like. A display controller 717 may also be provided, for converting data 707 stored in the memory 703 into text, graphics, and/or moving images (as appropriate) shown on the display device 715.

The various components of the computer system 700 may be coupled together by one or more buses, which may include a power bus, a control signal bus, a status signal bus, a data bus, etc. For the sake of clarity, the various buses are illustrated in FIG. 7 as a bus system 719.

### INDUSTRIAL APPLICABILITY

This disclosure generally relates to generating and providing exercise and health recommendations for a user. An exercise recommendation system may receive exercise information for a user. The exercise recommendation system may identify various exercise activity trends and/or habit information from the exercise information. For example, the exercise recommendation system may identify a missed workout. An exercise chatbot may interact with the user to collect additional information regarding the missed workout. For example, the exercise chatbot may prepare or generate a query and provide the query to the user. The user may provide a response including additional information related to the missed workout. In some embodiments, the exercise chatbot engages with the user, such as by providing one or more follow-up queries. A recommendation model may analyze the additional information and prepare one or more recommendations for the user. The recommendations may be directed to facilitating the user not missing future workouts. In this manner, the exercise recommendation system may provide improved recommendations tailored to the user. This may help to improve the user's consistency in performing exercise activities.

In some embodiments, the exercise recommendation system analyzes the health habits of the user. For example, the exercise recommendation system may receive and analyze historical health information for the user. A health habit model may analyze the historical health information for the user. The health habit model may prepare one or more recommendations to improve the user's health habits. For example, the health habit model may prepare a recommendation to instruct the user on actions that he or she may perform to improve health habits. The exercise recommendation system may provide the user with the recommendation. In some embodiments, the exercise recommendation system follows up on the user's exercise information to determine whether the user has adopted the recommendation and generated the desired habit. In this manner, the exercise recommendation system may continue to monitor the user's exercise information and provide recommendations to improve the user's health habits.

As illustrated by the foregoing discussion, the present disclosure utilizes a variety of terms to describe features and advantages of the exercise recommendation system. Additional detail is now provided regarding the meaning of such terms. For example, as used herein, the term “exercise information” (e.g., health information) refers to information related to health and/or exercise. In particular, the term exercise information may include information related to one or more exercise activities (e.g., workouts). For example, exercise information may include information related to the performance of the exercise activity, such as exercise activity type, exercise activity day (e.g., date, day of the week), exercise activity time of day, exercise device used, exercise device operating parameters (e.g., resistance, speed, incline level, weight setting), location of exercise device, exercise activity duration, training plan information, any other information related to the performance of the exercise activity, and combinations thereof. In some embodiments, exercise information includes user exercise information. For example, the exercise information may include heartrate information, electrocardiogram (EKG) information, blood sugar information, blood oxygen information, any other user exercise information, and combinations thereof. In some embodiments, exercise information includes user lifestyle or habit information. For example, user lifestyle or habit information may include sleep information (e.g., duration, time, quality), diet and nutrition information (e.g., food eaten, supplements taken, time of meals), work details, any other user lifestyle or habit information, goal information, and combinations thereof. In some embodiments, exercise information includes qualitative user exercise information. For example, qualitative user exercise information may include stress levels, pain levels, fatigue levels, attitude levels, motivation levels, any other qualitative user exercise information, and combinations thereof.

As used herein, a “foundation model” refers to an artificial intelligence (AI) or machine learning (ML) model that is trained to generate an output in response to an input based on a large dataset. The present disclosure may interchangeably refer to foundation models as AI models or ML models. A foundation model may be formed using a neural network having a significant number of parameters (e.g., billions of parameters). The foundation model may utilize the parameters to perform a task or otherwise generate an output based on an input. In one or more embodiments described herein, a foundation model is trained to generate a response to a query. In some implementations, a foundation model refers to a large language model (LLM). The foundation model be trained in any manner. For example, the foundation model may be trained on pattern recognition and text prediction. For example, the foundation model may be trained to predict the next word of a particular sentence or phrase. In one or more implementations described herein, the foundation model refers specifically to an LLM, though other types of foundation models may be used in generating responses to input queries.

As used herein, a chatbot may be a foundation model or an ML model that is trained in natural language algorithms to provide queries to a user and receive responses to the queries. The chatbot may be trained in natural language algorithms that may simulate a human interaction. For example, the chatbot may be trained to generate and present a query using syntax, verbs, nouns, and other grammatical elements to provide information and request information as a human being would. The chatbot may be trained to collect information based on an input dataset, such as the exercise information discussed herein. In some embodiments, the chatbot analyzes the input dataset, identify initial patterns in the input dataset, and request additional information to complete the pattern analysis. In some embodiments, the chatbot receives the pattern analysis from a different ML model, such as an exercise analysis model, health habit model, or other ML model. The chatbot may be interactive. For example, the chatbot may be trained to analyze the received response and generate additional content to provide the user. Such content may include recommendations, additional queries, motivational information, any other content, and combinations thereof.

As used herein, a recommendation model may refer to a foundation model that is trained to generate health or exercise recommendations based on an input dataset. The input dataset may include exercise information and/or historical exercise information. Historical exercise information may include any exercise information previously collected. In some embodiments, historical exercise information includes exercise information related to exercise activities prior to the most recent exercise activities. In some embodiments, historical exercise information includes daily exercise information for a period of time (e.g., one day, one week, one month, one year, multiple years). The recommendation model may be trained on a recommendation training dataset. The recommendation training dataset may include exercise information from people that exhibit positive exercise habits and/or have met previously set exercise goals or health habit goals. The recommendation model may receive the exercise information for a user and compare the user's exercise information, exercise goals, and health habit goals to the patterns identified by the recommendation model. The recommendation model may provide behavioral changes for the user to implement to meet his or her goals.

As used herein, an exercise recommendation may be used to refer to any recommendation to improve a user's health, including a recommendation to improve health habits and/or exercise consistency. For example, the exercise recommendation may include a change in behavior that may be associated with a user's health habits. In some examples, the exercise recommendation may include a change in environment. In some examples, the exercise recommendation may include any change in time of day for exercise, a change in exercise activity duration, a change in exercise activity intensity, a change in trainer, a change in exercise activity location, any other change in behavior or environment, and combinations thereof.

In some embodiments, the exercise recommendation is informational. For example, the exercise recommendation may include environmental information, habit stacking, a reward cycle, educational material, any other information, and combinations thereof. The environmental information may include any type of environmental information, such as locations for exercise, exercise apparel, people to exercise with, music type, music playlists, trainer ID, any other environmental information, and combinations thereof. In some examples, the educational material may include process-oriented education (e.g., changes in a series of activities leading to and/or during an exercise activity). In some examples, the educational material may include consistency education.

As used herein, the term “health habits” may refer to actions that are consistently performed that result in outcomes associated with positive health. Health habits may refer to any type of behavior, such as sleep-related actions, exercise activity-related actions, food and meal-related actions, any other health actions, and combinations thereof. In some embodiments, health habits are different for different people. In some embodiments, the recommendation model identifies commonalities in the exercise information and health habits, and provide recommendations to change the health habits. A health habit model may be an ML model that may be trained on trainer health habits of trainers.

An exercise recommendation system may generate and prove exercise and health recommendations to the user based on exercise information collected by and from the user. The exercise recommendation system may collect exercise and health information from the user using one or more user devices. The user devices may include any type of user device. For example, the user devices may include exercise devices, such as treadmills, elliptical machines, stationary bicycles, rowers, cable exercise devices, weight devices, any other exercise device, and combinations thereof. The user devices may include one or more wearable devices. The wearable devices may be any type of wearable device, such as a smartwatch, a smart ring, a sleep monitor, a heartrate monitor, any other type of wearable device, and combinations thereof. The user devices may include a computing device, such as a mobile device, a smartphone, a tablet, a laptop computer, a desktop computer, a server computer, any other computing device, and combinations thereof. In some embodiments, the user devices include any other type of device, such as medical devices, GPS trackers, pedometers, any other user devices, and combinations thereof.

The exercise recommendation system may provide the exercise information with one or more ML models, such as a missed activity identifier, a health habit model, a recommendation model, and an exercise chatbot. The ML models may analyze the exercise information to provide recommendations to the user. For example, the missed activity identifier may analyze the exercise information and identify a missed exercise activity. To identify the missed activity, the missed activity identifier may identify patterns of exercise activities and determine that an exercise activity was not performed based on the identified pattern and the exercise information. In some examples, the health habit model may analyze the exercise information to identify health habits for the user. The health habit model may compare the user's health habits to health habits identified by the health habit model. The exercise recommendation system may apply the recommendation model to generate exercise recommendations. For example, the recommendation model may receive the exercise information, the missed activity, and the health habit model and prepare or generate one or more recommendations for the user to implement or change a behavior to improve the user's health and/or exercise consistency. This may help the user to make and attain his or her health and exercise goals.

The exercise chatbot may interact with the user. For example, the exercise chatbot may include a query generator that generates and provides queries to the user to request additional information from the user. In some embodiments, the exercise chatbot includes an exercise information analyzer that may analyze the exercise information and/or the additional information from the user.

The query generator may generate queries to provide to the user. The query generator may generate the queries based on any information. For example, the query generator may generate the queries based on the exercise information. In some examples, the query generator may generate the queries based on missed activity information received from the missed activity identifier. In some examples, the query generator may generate the queries based on health habit information received from the health habit model. In some examples, the query generator may generate the queries based on recommendation information from the recommendation model. In some examples, the query generator may generate the queries based on pre-determined lines of inquiry. The pre-determined lines of inquiry may include goals, attitudes, interests, access to facilities, access or ownership of equipment, time availability, past successes, any other pre-determined line of inquiry, and combinations thereof.

The user may interact with the exercise chatbot on one or more of the user devices. For example, the exercise chatbot may present the query to the user through one of the user devices (e.g., through text, images, and/or videos a display, through sound from a speaker). The exercise chatbot may provide a response to the query through the user device. The user may provide the response to the exercise chatbot in any manner. For example, the user may provide the response to the through text input, image input (e.g., pictures, illustrations), video input, sound input (e.g., voice commands, text-to-speech), any other input mechanism, and combinations thereof. In some examples, the user may provide the response by uploading or linking a dataset. For example, the user may include health information in a datafile and upload the datafile to the exercise chatbot. In some examples, the user may have recorded health information using a third-party application, and the user may provide a link, download, or other mechanism to access the recorded data from the third-party application.

In some embodiments, the user provides exercise information to the exercise chatbot that is the same as at least a portion of the exercise information collected by the user devices. This may allow the exercise chatbot to validate the exercise information and/or ensure that the exercise information provided by the user devices is accurate, complete, up-to-date, or representative of the user's actual exercise information.

In accordance with at least one embodiment of the present disclosure, the exercise chatbot requests additional exercise information from the user. The additional exercise information include exercise information that was not included in the originally provided exercise information. For example, the additional exercise information may include exercise information that the user did not previously record on the user devices. In some examples, the additional exercise information may include qualitative exercise information that was previously uncaptured. In some examples, the additional exercise information may include sleep quality, a cause of the missed exercise activity, schedule information, exercise activity quality information, exercise activity difficulty information, exercise information intensity information, habit information, motivational information, any other exercise information, and combinations thereof.

In some embodiments, the exercise chatbot requests the additional exercise information in the query. For example, the query generator may generate the query and request the additional exercise information. The exercise chatbot may provide the query to the user. The user may prepare and submit a response to the exercise chatbot with the additional exercise information.

In some embodiments, the exercise chatbot provides the additional information to any other part of the exercise recommendation system. For example, the exercise chatbot may provide the additional exercise information to the missed activity identifier, the health habit model, the recommendation model, any other portion of the exercise recommendation system, and combinations thereof. This may allow the exercise recommendation system to identify additional patterns or associations between the originally provided exercise information, the additional exercise information, the training plan information, any other information, and combinations thereof.

The query generator may generate the query for the additional information based on any parameter. For example, the query generator may generate the query based on a set of pre-questions. In some examples, the query generator may generate the query based on the originally provided exercise information. In some examples, the query generator may generate the query based on an analysis from the missed activity identifier, the health habit model, the recommendation model, any other portion of the exercise recommendation system, and combinations thereof.

In some embodiments, the exercise chatbot enters into a conversation with the user. For example, the exercise chatbot may receive the response including the additional exercise information from the user. The exercise information analyzer may analyze the received additional exercise information. Based on the analysis of the additional exercise information, the query generator may generate a follow-up query for the user asking for further exercise information. The follow-up query may be related to the received additional exercise information provided by the user as the response to the first query.

The exercise chatbot may send the follow-up query to the user, and the user may provide follow-up exercise information based on the follow-up query. In this manner, the user may enter into a chat or a conversation with the user. The exercise chatbot may provide any number of follow-up queries to the user, and the user may provide any number of responses. In this manner, the exercise chatbot may receive more exercise information with which the exercise recommendation system may prepare exercise recommendations.

In some embodiments, the follow-up query includes a response to a question from the user. For example, in the response to one or more of the queries, the user may provide a question to the exercise chatbot. The question may be any type of question, such as a request for additional information, a request for clarification, a health question related to health information, an exercise information question related to a particular piece of exercise information or set of exercise information, an exercise activity question related to a particular exercise activity, an exercise device question related to a particular exercise device, any other request for information, and combinations thereof.

In some embodiments, the follow-up query is processed or generated using natural-language processing. For example, the query generator may include an ML model trained to generate natural language responses. This may help to improve the responsiveness of the exercise chatbot and/or improve the engagement of the user with the exercise chatbot.

In some embodiments, the query generator generates the follow-up query based on information received from the missed activity identifier, the health habit model, and/or the recommendation model. For example, the user may provide additional exercise information based on a query from the exercise chatbot. The missed activity identifier may identify one or more potential explanations for a missed exercise activity, and the missed activity identifier may have the query generator generate a follow-up query to request additional from the user related to the potential explanation for the missed exercise activity. The exercise chatbot may provide the follow-up query to the user, and the user may provide the additional exercise information related to the follow-up query. This may allow the missed activity identifier to enhance its analysis of the exercise information to identify the potential explanation for the missed activity. In some examples, the recommendation model may compare the missed exercise activity with a previous missed exercise activity to identify common behaviors in the missed exercise activity and the previous missed exercise activity.

In some examples, the query generator may generate the follow-up query based on the health analysis by the health habit model. For example, the health habit model may identify a potential health habit from the exercise information and/or the health information provided by the user. The health habit model may request additional information, and the query generator may generate the follow-up query based on the requested additional information. Exercise chatbot may provide the follow-up query to the user based on the request for additional information. The user may provide the additional exercise information based on the follow-up query. This may allow the health habit model to enhance its analysis of the exercise information to identify one or more health habits of the user.

In some examples, the query generator may generate the follow-up query based on a recommendation analysis by the recommendation model. For example, the recommendation model may begin preparing an exercise recommendation. The recommendation model may request additional information about the user from the exercise chatbot. The query generator may generate the follow-up query based on the requested additional information. The user may provide the additional exercise information based on the follow-up query. This may allow the recommendation model to enhance its recommendation based on the additional exercise information received by the user.

In accordance with at least one embodiment of the present disclosure, the exercise chatbot presents the query to the user without any user input. For example, the exercise chatbot may proactively send the user the query without receiving a user question or request for information. In some examples, the exercise chatbot may proactively send the user the query based on one or more of the analyses of the missed activity identifier, the health habit model, the recommendation model, and combinations thereof. In some examples, the exercise chatbot may proactively present the user with the query at a pre-scheduled time. The pre-scheduled time may be based on any time. For example, the pre-scheduled time may include a time of day, a day of the week, a day of the month, any other pre-scheduled time, and combinations thereof. In some embodiments, the pre-scheduled time is based on a trigger event. For example, the pre-scheduled time may be based on after completing an exercise activity, in a period after a missed exercise activity, after identifying a health habit, after identifying an area for improvement of a health habit, any other trigger event, and combinations thereof.

As a specific, non-limiting example, the missed activity identifier may identify, using the exercise information, that the user has missed an activity. The missed activity identifier may provide the exercise information analyzer and/or the query generator the missed activity. In some examples, the missed activity identifier may provide the recommendation model with the missed exercise activity and the recommendation model may prepare a recommendation for the user. Upon receipt of the missed activity and/or the recommendation based on the missed activity, the exercise chatbot may generate a query. The query may include any information discussed herein, including a request for additional exercise information, motivational information, some or all of the recommendation by the recommendation model, a request for additional information by the missed activity identifier and/or the recommendation model, any other information, and combinations thereof. As discussed herein, the exercise chatbot may provide the query to the user and receive a response from the user. In some examples, the exercise chatbot may enter into a conversation with the user based on the missed activity. This may help the user to identify the missed activity and implement changes to his or her lifestyle to prevent the missing another activity.

As a specific, non-limiting example, the health habit model may receive exercise activity information (e.g., health habit information). The health habit model may identify one or more existing health habits or opportunities for making and/or improving of a health habit. The health habit model may provide the health habit and/or the opportunities to the exercise chatbot. In some examples, the health habit model may provide the health habit and/or the opportunities to the recommendation model, and the recommendation model may provide a recommendation to the exercise chatbot. The exercise chatbot may generate and/or present a query to the user, the query including the health habit and/or the recommendation. As discussed herein, the user may prepare a response. In some examples, the exercise chatbot may enter into a conversation with the user based on the health habit and/or opportunities. This may help the user to identify health habits and/or make or improve new health habits.

Each of an components of the exercise recommendation system can include software, hardware, or both. For example, the components can include one or more instructions stored on a computer-readable storage medium and executable by processors of one or more computing devices, such as a client device or server device. When executed by the one or more processors, the computer-executable instructions of the exercise recommendation system can cause the computing device(s) to perform the methods described herein. Alternatively, the components can include hardware, such as a special-purpose processing device to perform a certain function or group of functions. Alternatively, the components of the exercise recommendation system can include a combination of computer-executable instructions and hardware.

Furthermore, the components of the exercise recommendation system may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

The exercise recommendation system may include one or more user devices. The user may interact with the user devices to communicate with the exercise recommendation system. For example, the user may input exercise information into the user devices. In some examples, the user may use the user devices to collect exercise information. The user devices may include one or more sensors, and the sensors on the user devices may collect exercise information about the user. As discussed herein, the user devices may include any type of user device, including exercise devices, wearable devices, computing devices, any other user device, and combinations thereof.

The exercise recommendation system may further include an exercise chatbot. The exercise chatbot may communicate with the user regarding exercise information. For example, the exercise chatbot may present the user with queries. The user may respond to the queries to the exercise chatbot. As discussed herein, the exercise chatbot may provide multiple queries to the user and the user may respond to the multiple queries. In this manner, the exercise chatbot may hold a conversation or other back-and-forth engagement with the user.

The exercise chatbot may include a query generator. The query generator may be trained to generate queries for the user. For example, the query generator may be trained in natural-language processing to provide queries to the user in a manner that the user may easily understand and be comfortable engaging in. In some examples, the query generator may be trained on exercise information, as discussed herein.

The exercise chatbot may further include an exercise information analyzer. The exercise information analyzer may analyze exercise information, including exercise information received by the user. The exercise information analyzer may communicate with the query generator to prepare the queries.

The exercise recommendation system may further include a missed activity identifier. The missed activity identifier may receive the exercise information from the user devices and/or additional exercise information from the exercise chatbot. The missed activity identifier may identify whether the user missed an activity. If the user misses an activity, the missed activity identifier may communicate the missed activity to the exercise chatbot. The exercise chatbot may generate and present a query to the user based on the missed activity.

The exercise recommendation system may include a health habit model. The health habit model may receive the exercise information from the user devices and/or additional exercise information from the exercise chatbot. The health habit model may analyze the exercise information and identify health habits and/or opportunities for improved or new health habits. The health habit model may provide the health habits and/or the opportunities to the exercise chatbot. As discussed herein, the exercise chatbot may generate and present a query to the user based on the health habits and/or opportunities.

The exercise recommendation system may further include a recommendation model. The exercise recommendation system may apply the recommendation model to the exercise information. For example, the recommendation model may receive the exercise information from the user devices and/or additional exercise information from the exercise chatbot. In some embodiments the recommendation model receives exercise analyses from the missed activity identifier and/or the health habit model. The recommendation model may be trained to generate an exercise recommendation for the user. The recommendation model may provide the exercise recommendation to the user devices. In some examples, the recommendation model may provide the recommendation to the exercise chatbot and the exercise chatbot may provide the recommendation to the user as a query and/or as part of a conversation with the user.

In accordance with at least one embodiment of the present disclosure, a user provides exercise information to an exercise recommendation system from a user device. A missed activity identifier may receive the exercise information. The missed activity identifier may identify a missed activity in the exercise information. For example, the missed activity identifier may include an ML model that may be trained to identify whether the user has missed an activity.

An exercise chatbot may receive the exercise information and the missed activity. The exercise chatbot may analyze the missed activity and the exercise information and prepare a query for the user. The exercise chatbot may generate the query in any manner. For example, the exercise chatbot may generate the query requesting additional information based on the missed activity and/or the exercise information. The exercise chatbot may provide the query to the user at the user device. The user may prepare a response including additional exercise information. For example, the response may include the user's response to any questions or requests in the query.

The user device may provide the response to the exercise chatbot. As discussed herein, the exercise chatbot may receive the response and prepare a follow-up query. The exercise chatbot may provide the follow-up query to the user at the user device, and the user may provide a follow-up response or additional response to the exercise chatbot. This loop may be repeated any number of times until the exercise chatbot does not have any additional queries to provide the user. While embodiments of the present disclosure have discussed the exercise chatbot as providing the follow-up query in response to the response by the user, it should be understood that the exercise chatbot may provide more queries than the user provides responses. For example, the exercise chatbot may provide the follow-up query without a response from the user. In some examples, the user may provide more responses than the user receives queries.

In some embodiments, the user provides a response to the exercise chatbot without receiving a query. For example, the user provide the exercise chatbot with a question or a request for a recommendation and/or additional information. The user may provide the question or request as a response to a previous query. For example, the exercise chatbot may include a window having one or more text entry boxes. The window may include a history of the conversation with the user. The user may provide the question or request in the text entry box. The exercise chatbot may receive the question or request as a response. The exercise chatbot may then initiate a conversation with the user based on the user input.

The exercise chatbot may provide the additional information received by the user device to a recommendation model. The exercise recommendation system may apply the recommendation model to the exercise information. The recommendation model may further receive the additional exercise information from the user device. The additional exercise information may include the additional exercise information provided in the responses. The exercise chatbot provides the additional information to the recommendation model at any point in time. For example, the exercise chatbot may provide the additional exercise information to the recommendation model as soon as it receives a response from the user device. In some examples, the exercise chatbot may combine one or more responses and provide them to the additional exercise information to the recommendation model as a group or a bundle. This may help to reduce bandwidth of the exercise recommendation system.

As discussed herein, the exercise recommendation system may apply the recommendation model to the exercise information. The recommendation model may prepare a recommendation for the user. The recommendation may be based on the exercise information and/or the additional exercise information. As discussed herein, the recommendation may include a change in one or more behaviors that may help the user to not miss a future exercise activity, or to be more reliable or regular in attending future exercise activities. The recommendation model may send the recommendation to the user device.

In accordance with at least one embodiment of the present disclosure, after the user device receives the recommendation the user device transmits exercise information to the missed activity identifier, the exercise chatbot, and the recommendation model, and the techniques described herein are repeated. For example, the recommendation may include a recommendation to change a lifestyle action, such as an exercise time. The exercise information may include information about the exercise time of the user. The missed activity identifier may analyze the exercise information to determine whether the user missed an exercise activity based on the new exercise time. The exercise chatbot may provide the user with information regarding the new exercise time and the recommendation. In some embodiments, the missed activity identifier identifies a success, or identifies when the user successfully develops a habit or exhibits an improved pattern of behavior based on the recommendation.

In some examples, the exercise information may indicate that the recommendation was not effective at encouraging the user to not miss exercise activities. For example, the missed activity identifier may analyze the exercise information after the user receives the recommendation. The missed activity identifier may identify that the user missed another activity. In some embodiments, the missed activity identifier, the exercise chatbot, and/or the recommendation model identifies improvement or backsliding based on the exercise information and/or additional exercise information provided based on a query to the user. For example, the exercise recommendation system may apply the missed activity identifier and/or the recommendation model to the exercise information and/or the additional exercise information. The recommendation model may analyze the exercise information and/or the additional exercise information and prepare another recommendation to facilitate the user not missing an additional exercise activity, or for the user to be more consistent in exercising.

In accordance with at least one embodiment of the present disclosure, a user provides exercise information to an exercise recommendation system from a user device. A health habit model may receive the exercise information. The health habit model may identify health habits in the exercise information. For example, the health habit model may include an ML model that may be trained to identify patterns of behavior correlated with health outcomes.

The health habit model may provide health habits to an exercise chatbot. The exercise chatbot may further receive the exercise information from the user device. Based on the health habits and/or the exercise information, the exercise chatbot may generate and present a query to the user at the user device. The user may receive the query and send a response including additional exercise information to the exercise chatbot. The exercise chatbot may provide the additional exercise information to a recommendation model.

The recommendation model may receive the additional exercise information. In some embodiments, the recommendation model receives the exercise information from the user device. The recommendation model may prepare an exercise recommendation for the user. The recommendation model may send the exercise recommendation to the user device. As discussed herein, the exercise recommendation may include any recommendation. For example, the exercise recommendation may include a recommendation for meal planning. This may help the user to prepare meals ahead of time and encourage health diet habits.

In accordance with at least one embodiment of the present disclosure, after the user receives the exercise recommendation, the health habit model receives exercise information. The health habit model may identify whether the exercise recommendation has been implemented. The techniques described herein may be applied to the exercise information after receiving and/or implementing the exercise recommendation. This may facilitate the health habit model, the exercise chatbot, and/or the recommendation model understanding whether the user implemented the exercise recommendation. The exercise chatbot may engage with the user regarding whether the user implemented the exercise recommendation. For example, the exercise chatbot may provide encouragement and/or follow-up questions regarding the implementation of the exercise recommendation. In some examples, the recommendation model may prepare additional or adjusted recommendations to further improve the user's health habits.

The following disclosure and the examples provide a number of different methods, systems, devices, and computer-readable media of the exercise recommendation system. In addition to the foregoing, one or more embodiments can also be described in terms of flowcharts comprising acts for accomplishing a particular result. The systems and methods described herein may be performed with more or fewer acts. Further, the acts may be performed in differing orders. Additionally, the acts described herein may be repeated or performed in parallel with one another or parallel with different instances of the same or similar acts.

The following acts can be performed as part of a method. Alternatively, a computer-readable medium can comprise instructions that, when executed by one or more processors, cause a computing device to perform the described acts. In some embodiments, a system can perform the described acts.

An exercise recommendation system may receive exercise information for a user. Based on the exercise information the exercise recommendation system may identify a missed exercise activity. Based on the missed exercise activity, and using an exercise chatbot, the exercise recommendation system may present the user with a query. The query may ask for additional information related to the missed exercise activity. The exercise recommendation system may receive a response to the query from the user. The exercise recommendation system may apply a recommendation model to the response and/or the exercise information to generate an exercise recommendation.

An exercise recommendation system may receive exercise information for a user. Based on the exercise information, the exercise recommendation system may provide the user with a query. The query may ask for health information. The exercise recommendation system may receive health information from the user. The health information include additional exercise information for the user. The exercise recommendation system may apply a health habit model to the health information. The heath habit model may identify a health habit for the user. The exercise recommendation system may generate, using the health habit model and/or a recommendation model, a recommendation for the user. The recommendation may be to improve the health habit of the user. The exercise recommendation system may then present the recommendation to the user.

The following discussion describes certain components that may be included within a computer system. One or more computer systems may be used to implement the various devices, components, and systems described herein.

The computer system includes a processor. The processor may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor may be referred to as a central processing unit (CPU). Although just a single processor is described herein, in an alternative configuration, a combination of processors (e.g., an ARM and DSP) could be used.

The computer system also includes memory in electronic communication with the processor. The memory may be any electronic component capable of storing electronic information. For example, the memory may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions and data may be stored in the memory. The instructions may be executable by the processor to implement some or all of the functionality disclosed herein. Executing the instructions may involve the use of the data that is stored in the memory. Any of the various examples of modules and components described herein may be implemented, partially or wholly, as instructions stored in memory and executed by the processor. Any of the various examples of data described herein may be among the data that is stored in memory and used during execution of the instructions by the processor.

A computer system may also include one or more communication interfaces for communicating with other electronic devices. The communication interface(s) may be based on wired communication technology, wireless communication technology, or both. Some examples of communication interfaces include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system may also include one or more input devices and one or more output devices. Some examples of input devices include a keyboard, mouse, microphone, remote control device, button, joystick, trackball, touchpad, and lightpen. Some examples of output devices include a speaker and a printer. One specific type of output device that is typically included in a computer system is a display device. Display devices used with embodiments disclosed herein may utilize any suitable image projection technology, such as liquid crystal display (LCD), light-emitting diode (LED), gas plasma, electroluminescence, or the like. A display controller may also be provided, for converting data stored in the memory into text, graphics, and/or moving images (as appropriate) shown on the display device.

The various components of the computer system may be coupled together by one or more buses, which may include a power bus, a control signal bus, a status signal bus, a data bus, etc. For the sake of clarity, the various buses described herein as a bus system.

Following are sections in accordance with at least one embodiment of the present disclosure:

A1. A method for providing exercise recommendations, the method comprising:
receiving exercise information for a user;
based on the exercise information, identifying a missed exercise activity;
based on the missed exercise activity and using an exercise chatbot, presenting the user with a query, the query including a request for additional information related to the missed exercise activity;
receiving a response to the query; and
applying a recommendation model to the response to generate an exercise recommendation.

A2. The method of section A1, wherein the exercise information includes historical exercise information.

A3. The method of section A2, wherein the historical exercise information includes daily exercise information for a period of time.

A4. The method of section A3, wherein the period of time includes one week.

A5. The method of any of sections A1-A4, wherein the exercise information includes an exercise activity type.

A6. The method of any of sections A1-A5, wherein the exercise information includes an exercise activity day and time.

A7. The method of any of sections A1-A6, wherein the exercise information includes heartrate.

A8. The method of any of sections A1-A7, wherein the exercise information includes training plan information.

A9. The method of any of sections A1-A8, wherein applying the recommendation model includes comparing the missed exercise activity with a previous missed exercise activity.

A10. The method of section A9, wherein applying the recommendation model includes identifying common behaviors between the missed exercise activity and the previous missed exercise activity.

A11. The method of any of sections A1-A10, wherein the exercise recommendation includes a change in time of day for exercise.

A12. The method of any of sections A1-A11, wherein the exercise recommendation includes a change in exercise activity type.

A13. The method of any of sections A1-A12, wherein the exercise recommendation includes a change in exercise activity duration.

A14. The method of any of sections A1-A13, wherein the exercise recommendation includes a change in exercise activity intensity.

A15. The method of any of sections A1-A14, wherein the exercise recommendation includes a change in trainer.

A16. The method of any of sections A1-A15, wherein the exercise recommendation includes a change in exercise activity location.

A17. The method of any of sections A1-A16, wherein presenting the user with the query includes presenting the user with the query without user input by the user.

A18. The method of any of sections A1-A17, wherein presenting the user with the query includes presenting the user with the query based on user input by the user.

A19. The method of any of sections A1-A18, wherein presenting the user with the query includes presenting the user with the query at a pre-scheduled time.

A20. The method of section A19, wherein the pre-scheduled time is after completing an exercise activity.

A21. The method of section A19 or sections A20, wherein the pre-scheduled time is a time of day.

A22. The method of any of sections A19-A21, wherein the pre-scheduled time is a day of the week.

A23. The method of any of sections A19-A22, wherein the pre-scheduled time is a period after the missed exercise activity.

A24. The method of any of sections A1-A23, wherein the additional information includes sleep quality.

A25. The method of any of sections A1-A24, wherein the additional information includes a cause of the missed exercise activity.

A26. The method of any of sections A1-A25, wherein the additional information includes schedule information.

A27. The method of any of sections A1-A26, wherein the additional information includes exercise activity quality information.

A28. The method of any of sections A1-A27, wherein the additional information includes exercise activity difficulty information.

A29. The method of any of sections A1-A28, wherein the additional information includes habit information.

A30. The method of any of sections A1-A29, wherein the query includes motivational information.

A31. The method of any of sections A1-A30, further comprising:
applying the exercise chatbot to the response; and
presenting the user with a follow-up query requesting for additional information related to the response and the missed exercise activity.

A32. The method of any of sections A1-A31, further comprising applying a health habit model to the exercise information.

A33. The method of section A32, wherein applying the exercise recommendation includes a health recommendation based on the health habit model.

B1. A method for exercise recommendations, the method comprising:
receiving exercise information for a user;
based on the exercise information, providing the user with a query, the query requesting health information from the user;
applying a health habit model to the health information, the health habit model identifying a health habit for the user;
generating, using the health habit model, a recommendation to improve the health habit; and
presenting the recommendation to the user.

B2. The method of section B1, wherein the health habit model includes a large language model (LLM).

B3. The method of section B1 or section B2, wherein the health habit model is trained on trainer health habits of trainers.

B4. The method of any of sections B1-B3, wherein the recommendation includes environmental information.

B5. The method of any of sections B1-B4, wherein the recommendation includes habit stacking.

B6. The method of any of sections B1-B5, wherein the recommendation includes a reward cycle.

B7. The method of any of sections B1-B6, wherein the recommendation includes educational material.

B8. The method of section B7, wherein the educational material includes process-oriented education.

B9. The method of section B7 or section B8, wherein the educational material includes consistency education.

B10. The method of any of sections B1-B9, wherein receiving the health information for the user includes receiving goal information for the user.

C1. Any device, apparatus, system, kit, component, or subcomponent as illustrated or described, or method of manufacture or use thereof.

D1. A method having any or each permutation of features recited in sections A1-A33 and B1-B10.

E1. An assembly/system/device having any or each permutation of features recited in A1-A33 and B1-B10.

F1. Any system, assembly, component, subcomponent, process, element, or portion thereof, as described or illustrated.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for providing exercise recommendations, comprising:
receiving exercise information for a user;
identifying, based at least in part on the exercise information, a missed exercise activity;
presenting, based at least in part on the missed exercise activity and using an exercise chatbot, the user with a query, the query comprising a request for additional information related to the missed exercise activity;
receiving a response to the query; and
applying a recommendation model to the response to generate an exercise recommendation.

2. The method of claim 1, wherein the exercise chatbot comprises a large language model (LLM).

3. The method of claim 1, wherein the exercise information comprises historical exercise information of the user.

4. The method of claim 1, wherein the exercise information comprises an exercise activity type, an exercise activity day and time, a heartrate of the user, training plan information, or any combination thereof.

5. The method of claim 1, wherein applying the recommendation model comprises:
comparing the missed exercise activity with a previous missed exercise activity.

6. The method of claim 5, wherein applying the recommendation model further comprises:
identifying common behaviors between the missed exercise activity and the previous missed exercise activity.

7. The method of claim 1, wherein the exercise recommendation comprises a change in time of day for exercise, a change in exercise activity type, a change in exercise activity duration, a change in exercise activity intensity, a change in trainer, a change in exercise activity location, or any combination thereof.

8. The method of claim 1, wherein presenting the user with the query comprises:
presenting the user with the query without user input by the user.

9. The method of claim 1, wherein presenting the user with the query comprises:
presenting the user with the query at a pre-scheduled time.

10. The method of claim 9, wherein the pre-scheduled time is based at least in part on a trigger event.

11. The method of claim 1, wherein the additional information comprises additional exercise information not included in the exercise information.

12. The method of claim 1, wherein the query comprises additional content.

13. The method of claim 1, further comprising:
applying the exercise chatbot to the response; and
presenting the user with a follow-up query requesting for additional information related to the response and the missed exercise activity.

14. A method for exercise recommendations, comprising:
receiving exercise information for a user;
providing, based at least in part on on the exercise information, the user with a query, the query requesting health information from the user;
applying a health habit model to the health information, the health habit model identifying a health habit for the user;
generating, using the health habit model, a recommendation to improve the health habit; and
presenting the recommendation to the user.

15. The method of claim 14, wherein the health habit model comprises a large language model (LLM).

16. The method of claim 14, wherein the health habit model is trained on trainer health habits of trainers.

17. The method of claim 14, wherein the recommendation comprises environmental information, habit stacking, a reward cycle, or any combination thereof.

18. The method of claim 14, wherein the recommendation comprises educational material.

19. The method of claim 18, wherein the educational material comprises process-oriented education, consistency education, or both.

20. An exercise recommendation system, comprising:
one or more processors and one or more memories, the one or more memories comprising instructions that, when executed by the one or more processors, cause the exercise recommendation system to:
receive exercise information for a user;
identify, based at least in part on the exercise information, a missed exercise activity;
present, based at least in part on the missed exercise activity and using an exercise chatbot, the user with a query, the query comprising a request for additional information related to the missed exercise activity;
receive a response to the query; and
apply a recommendation model to the response to generate an exercise recommendation.
